(19) United States
(12) Patent Application Publication
(10) Pub. No.: US 2023/0046717 A1
(43) Pub. Date: Feb. 16, 2023

(54) Light Shield Structure

(71) Applicant: XIAMEN BRI ENVIRONMENTAL INDUSTRY CO., LTD., Xiamen (CN)

(72) Inventor: Huoju Su, Xiamen (CN)

(21) Appl. No.: 17/746,999

(22) Filed: May 18, 2022

(30) Foreign Application Priority Data
Aug. 12, 2021 (CN) 202121886948.3

(51) Int. Cl. F21V 9/06; A61L 9/20

(52) U.S. Cl. CPC F21V 9/06; A61L 9/20

(57) ABSTRACT

A light shield structure is disposed between an ultraviolet lamp and a filter screen of an air purifier, and comprises a first light shield element and a second light shield element. The first light shield element is disposed on an inner wall of the second light shield element. The second light shield element is provided with air inlets, and the first light shield element is staggered with the second light shield element, so that the air inlets are completely blocked by the first light shield element in a radial direction. An air inlet channel is formed between the first light shield element and the second light shield element. Under the precondition that the air inlet channel is formed by the first light shield element and the second light shield element, ultraviolet light can be completely shielded, so that the service life of the filter screen is prolonged.

### CROSS REFERENCE TO RELATED APPLICATIONS

The present application claims the benefit of Chinese Pat. App No. 2021218869483 filed on Aug. 12, 2021, the contents of which are incorporated herein by reference in their entirety.

### TECHNICAL FIELD

The invention relates to the field of purifiers, in particular to a light shield structure.

### DESCRIPTION OF RELATED ART

Due to the outbreak of viruses, the demand for air purifiers internally provided with an ultraviolet lamp is becoming ever urgent. In existing air purifiers, a filter screen made of PP or PET is not resistant to ultraviolet irradiation, and may be aged and covered with dust after being exposed to ultraviolet irradiation for a long time, thus doing harm to human health. In view of this, a light shield structure is additionally disposed between the filter screen and the ultraviolet lamp in the prior art. However, due to the fact that a large gap is reserved in the light shield structure to allow air to flow in smoothly, ultraviolet light cannot be completely shielded, and the filter screen may still be exposed to ultraviolet light. Or, in order to completely protect the filter screen against ultraviolet light, a small air inlet is reserved in the light shield structure, but the air input is small, so although the filter screen is completely shielded against ultraviolet light, the air input is greatly decreased, thus reducing the purification efficiency of the air purifier.

### BRIEF SUMMARY OF THE INVENTION

The technical issue to be settled by the invention is to provide a light shield structure that can protect a filter screen against ultraviolet irradiation under the precondition that the air input is guaranteed.

To settle the above technical issue, the technical solution adopted by the invention is as follows:

A light shield structure is disposed between an ultraviolet lamp and a filter screen of an air purifier, and comprises a first light shield element and a second light shield element;

The first light shield element is disposed on an inner wall of the second light shield element;

The second light shield element is provided with air inlets, the first light shield element is provided with air outlets, and the air inlets are staggered with the air outlets, so that the air outlets and the air inlets are completely blocked on at least one side in a radial direction; and an air inlet channel is formed between the first light shield element and the second light shield element.

Further, first guide slopes and second guide slopes are disposed on an outer wall of the first light shield element, and a first angle which is less than 180° is formed between the first guide slopes and sides, away from the second light shield element, of the second guide slopes.

Further, third guide slopes and fourth guide slopes are disposed on an outer wall of the second light shield element, and a second angle which is less than 180° is formed between the third guide slopes and sides, facing the first light shield element, of the fourth guide slopes.

Further, the first light shield element is arranged on one side of the air inlets and is centered in a circumferential direction.

Further, the light shield structure further comprises multiple third light shield elements;

The multiple third light shield elements are regularly disposed on the inner wall of the second light shield element, and an air outlet channel is formed between every two adjacent third light shield elements;

The air inlets and the air outlet channels opposite to the air inlets in the radial direction are communicated through the air inlet channel;

The first light shield element is arranged in the air inlet channel and is centered in the circumferential direction.

Further, a fifth guide slope and a sixth guide slope are disposed on an outer wall of each third light shield element, and a third angle which is less than 180° is formed between the fifth guide slope and a side, away from the second light shield element, of the sixth guide slope.

Further, the first light shield element and the second light shield element are formed integrally.

Further, the third light shield elements and the second light shield element are formed integrally.

The invention has the following beneficial effects: the first light shield element and the second light shield element are staggered to form the air inlet channel, so that the filter screen is protected against ultraviolet light under the precondition that air flows into the second light shield element smoothly, the service life of the filter screen is prolonged, and the filter screen will not be aged within a short time.

### BRIEF DESCRIPTION OF THE SEVERAL VIEWS OF THE DRAWINGS

FIG. 1 is an exploded view of an air purifier of the invention;

FIG. 2 is a front view of the longitudinal section of the air purifier of the invention;

FIG. 3 is a structural diagram of a light shield structure, an ultraviolet lamp and a filter screen in Embodiment 2 of the invention;

FIG. 4 is a structural diagram of a light shield structure in Embodiment 3 of the invention;

FIG. 5 is a sectional view of FIG. 4;

FIG. 6 is a structural diagram of a light shield structure in Embodiment 4 of the invention;

FIG. 7 is a sectional view of FIG. 6.

### Reference Signs

1, ultraviolet lamp;
2, filter screen;
3, first light shield element; 31, first guide slope; 32, second guide slope; 33, air outlet;
4, second light shield element; 41, air inlet; 42, third guide slope; 43, fourth guide slope;
5, air inlet channel;
6, third light shield element; 61, fifth guide slope; 62, sixth guide slope;
7, air outlet channel.

### DETAILED DESCRIPTION OF THE INVENTION

The technical contents, purposes and effects of the invention will be described in detail below in conjunction with embodiments and accompanying drawings.

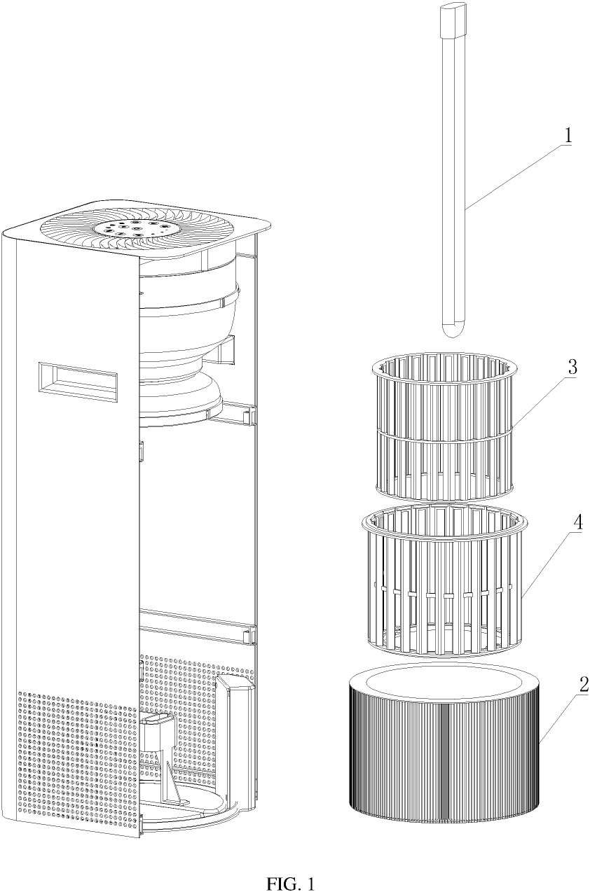

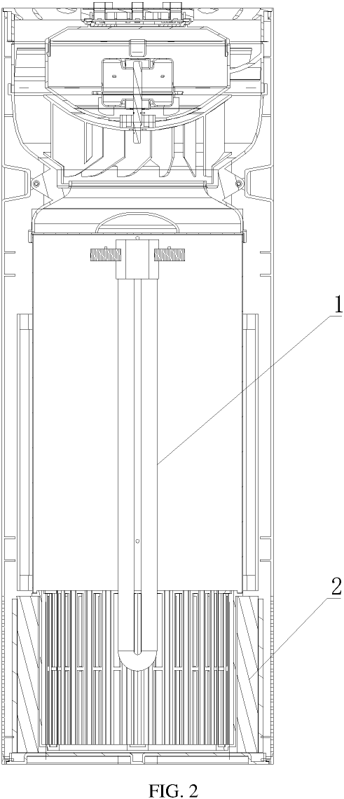

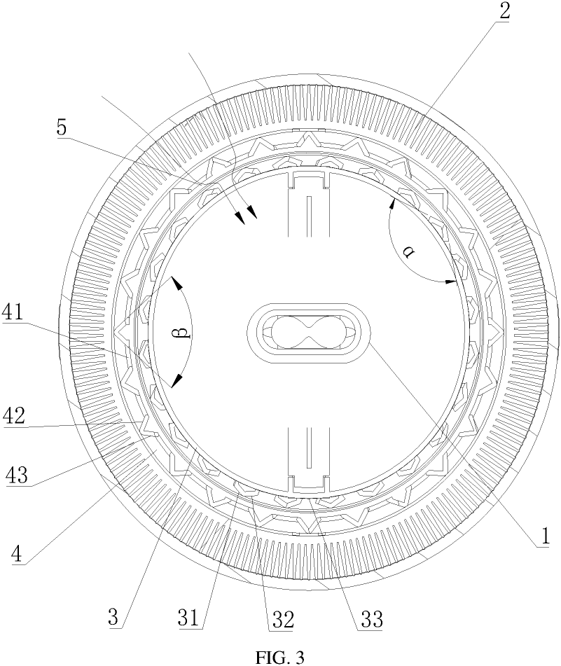

As shown in FIG. 1-FIG. 7, a light shield structure is disposed between an ultraviolet lamp and a filter screen of an air purifier and comprises a first light shield element and a second light shield element;

The first light shield element is disposed on an inner wall of the second light shield element;

The second light shield element is provided with air inlets; the first light shield element is staggered with the second light shield element, so that the first light shield element completely blocks the air inlets in a radial direction; and an air inlet channel is formed between the first light shield element and the second light shield element.

The working principle of the invention is as follows:

The first light shield element and the second light shield element are staggered, so that the air inlets are blocked by the first light shield element in the radial direction; and the air inlet channel is reserved, so that the air input is not decreased, and the filter screen is protected against ultraviolet light.

From the above description, the invention has the following beneficial effects: the first light shield element and the second light shield element are staggered to form the air inlet channel, so that the filter screen is protected against ultraviolet light under the precondition that air flows into the second light shield element smoothly, the service life of the filter screen is prolonged, and the filter screen will not be aged or covered with dust within a short time.

Further, multiple light shield bodies are disposed on an outer wall of the first light shield element, and an air outlet is formed between every two adjacent light shield bodies;

The air inlets and the corresponding air outlets are communicated through the air inlet channel.

From the above description, the light shield bodies are used to seal the air inlets, and the air outlets are formed between the adjacent light shield bodies and are communicated with the air inlets to form the air inlet channel, so that air can enter the air purifier smoothly and can be sterilized by the ultraviolet lamp.

Further, each light shield body comprises a first guide slope and a second guide slope, and a first angle which is less than 180° is formed between the first guide slope and a side, away from the second light shield element, of the second guide slope.

From the above description, the first guide slopes and the second guide slopes are arranged, and the first angle is formed between the first guide slopes and the second guide slopes, so that an air flow in the air inlet channel can be divided and guided to enter the air outlets along the first guide slopes and the second guide slopes, thus reducing the air intake resistance and ensuring the air intake efficiency.

Further, third guide slopes and fourth guide slopes are disposed on an outer wall of the second light shield element, and a second angle which is less than 180° is formed between the third guide slopes and sides, facing the first light shield element, of the fourth guide slopes.

From the above description, the third guide slopes and the fourth guide slopes are arranged, and the second angle is formed between the third guide slopes and the fourth guide slopes, so that an air flow in the air inlet channel can be divided and guided to enter the air inlets along the third guide slopes and the fourth guide slopes, thus reducing the air intake resistance and ensuring the air intake efficiency.

Further, multiple first light shield elements are regularly disposed on the inner wall of the second light shield element;

The first light shield element is arranged on one side of the air inlets and is centered in a circumferential direction.

From the above description, the multiple first light shield elements are arranged, and each first light shield element is centered, so that the light shield effect is guaranteed.

Further, the light shield structure further comprises multiple third light shield elements;

The multiple third light shield elements are regularly disposed on the inner wall of the second light shield element, and an air outlet channel is formed between every two adjacent third light shield elements;

The air inlets and the air outlet channels opposite to the air inlets in the radial direction are communicated through the air inlet channel;

The first light shield elements are arranged in the air inlet channel and are centered in the circumferential direction.

From the above description, the third light shield elements are arranged to seal gaps between the first light shield elements and the inner wall of the air inlet channel, so that ultraviolet light is completely shielded.

Further, a fifth guide slope and a sixth guide slope are disposed on an outer wall of each third light shield element, and a third angle which is less than 180° is formed between the fifth guide slope and a side, away from the second light shield element, of the sixth guide slope.

From the above description, the fifth guide slopes and the sixth guide slopes are arranged, and the third angle is formed between the fifth guide slopes and the sixth guide slopes, so that an air flow in the air inlet channel is divided and guided to enter the air outlet channels along the fifth guide slopes and the sixth guide slopes, thus reducing the air intake resistance and ensuring the air intake efficiency.

Further, the first light shield element and the second light shield element are formed integrally.

From the above description, the first light shield element and the second light shield element are formed integrally, so that the stability of the light shield structure is improved.

Further, the third light shield elements and the second light shield element are formed integrally.

From the above description, the third light shield elements and the second light shield element are formed integrally, so that the stability of the light shield structure is improved.

### Embodiment 1

Referring to FIGS. 1-FIGS. 7, a light shield structure is disposed between an ultraviolet lamp 1 and the filter screen 2 of an air purifier, and comprises a first light shield element 3 and a second light shield element 4, wherein the first light shield element 3 is disposed on an inner wall of the second light shield element 4; the second light shield element 4 is provided with air inlets 41, the first light shield element 3 is provided with air outlets 33, and the air inlets 41 are staggered with the air outlets 33, so that the air outlets 33 and the air inlets 41 are completely blocked on at least one side in a radial direction; and an air inlet channel 5 is formed between the first light shield element 3 and the second light shield element 4.

### Embodiment 2

Referring to FIG. 1-FIG. 3, on the basis of Embodiment 1, first guide slopes 31 and second guide slopes 32 are disposed on an outer wall of the first light shield element 3, and a first angle α which is less than 180° is formed between the first guide slopes 31 and sides, away from the second light shield element 4, of the second guide slopes 32. Preferably, the first angle α is 127°, and the air inlets 41 and the corresponding air outlets 33 are communicated through the air inlet channel 5. Specifically, the first light shield element 3 and the second light shield element 4 are both of a circular structure, and the first light shield element 3 is sleeved with the second light shield element 4.

Referring to FIG. 1-FIG. 3, third guide slopes 42 and fourth guide slopes 43 are disposed on an outer wall of the second light shield element 4, and a second angle β which is less than 180° is formed between the third guide slopes 42 and sides, facing the first light shield element 3, of the fourth guide slopes 43. Preferably, the second angle β is 84°.

### Embodiment 3

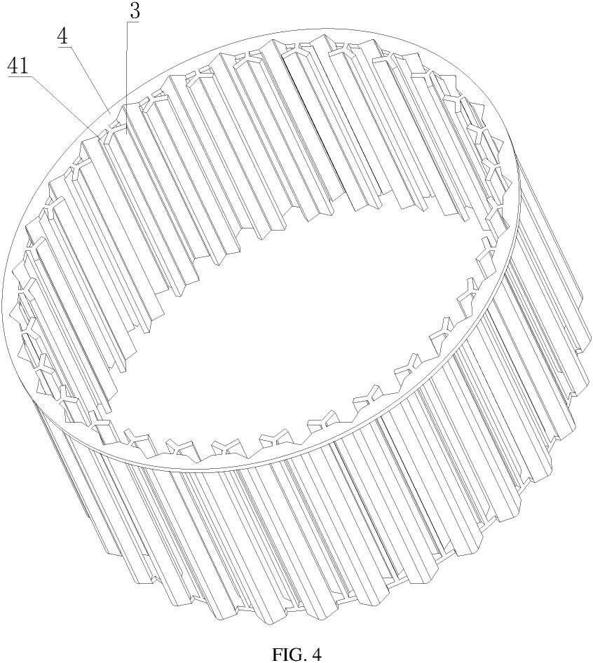

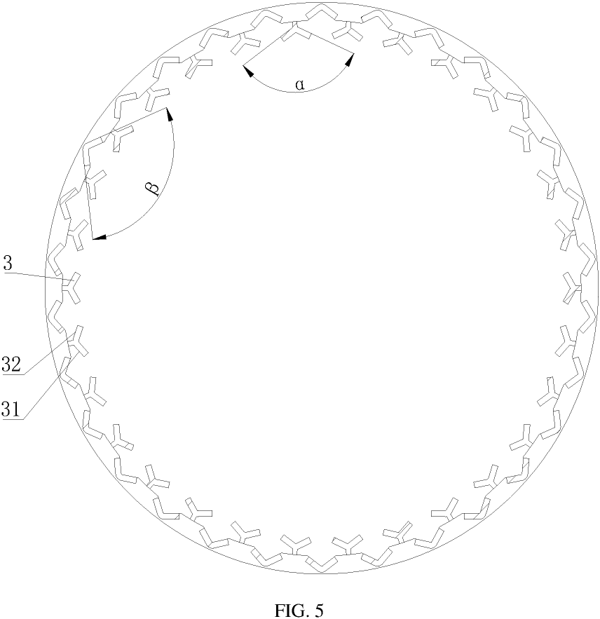

Referring to FIG. 4 and FIG. 5, on the basis of Embodiment 2, first light shield elements 3 of a different structure are arranged. The first light shield elements 3 are arranged on one side of the air inlets 41 and are centered in a circumferential direction. Specifically, the first light shield elements 3 are disposed on the inner wall of the second light shield element 4 and are able to completely shield light entering the air inlets 41 in a radial direction. In this embodiment, each first light shield element 3 is provided with a first guide slope 31 and a second guide slope 32, and a first angle α is formed between the first guide slope 31 and the second guide slope 32; and preferably, the first angle α is 119°, and the second angle β is 107°.

Preferably, the first light shield elements 3 and the second light shield element 4 are formed integrally.

### Embodiment 4

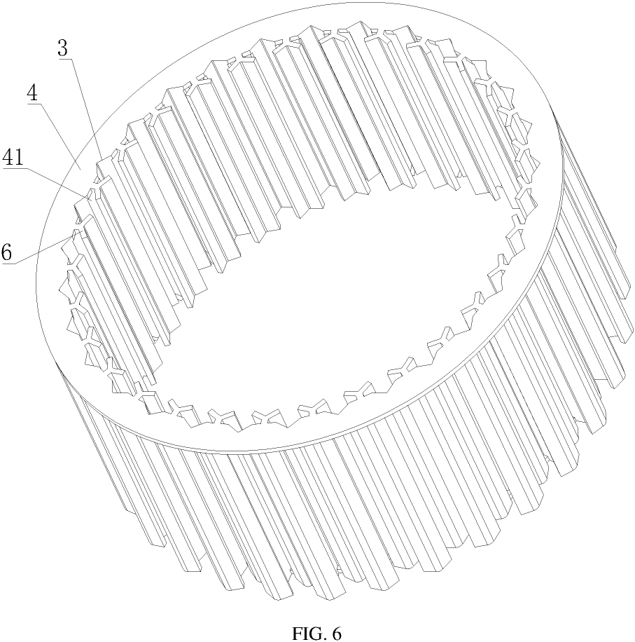

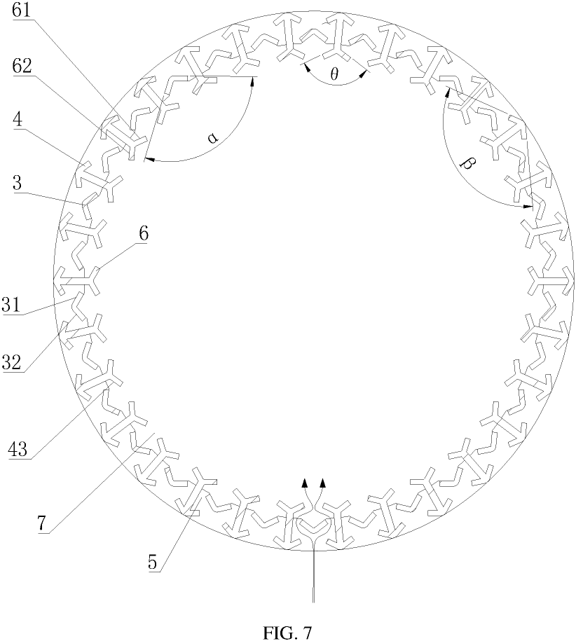

Referring to FIG. 6 and FIG. 7, on the basis of Embodiment 2, first light shield elements 3 of a different structure are arranged, and the light shield structure also comprises multiple third light shield elements 6; the multiple third light shield elements 6 are regularly disposed on the inner wall of the second light shield element 4, and an air outlet channel 7 is formed between every two adjacent third light shield elements 6; the air inlets 41 and the air outlet channels 7 opposite to the air inlets 41 in the radial direction are communicated through the air inlet channel 5; and the first light shield elements 3 are arranged in the air inlet channel 5 and are centered in the circumferential direction.

Referring to FIG. 6 and FIG. 7, a fifth guide slope 61 and a sixth guide slope 62 are disposed on an outer wall of each third light shield element 6, and a third angle θ which is less than 180° is formed between the fifth guide slopes 61 and sides, away from the second light shield element 4, of the sixth guide slopes 62. In this embodiment, each first light shield element 3 is provided with a first light guide slope 31 and a second light guide slope 32, and a first angle α is formed between the first light guide slope 31 and the second guide slope 32; and preferably, the first angle α is 107°, the second angle β is 119°, and the third angle θ is 119°.

Preferably, the first light shield elements 3 and the second light shield element 4 are formed integrally, and the third light shield elements 6 and the second light shield element 4 are formed integrally.

To sum up, three different light structures may be formed by the first light shield element, the second light shield element and the third light shield element, and under the precondition that the air inlet channel is reserved, light entering each air inlet may be shielded in the radial direction through the first light shield element and the second light shield element or through the first light shield element, the second light shield element and the third light shield elements, so that the filter screen is protected against ultraviolet light, and the service life of the filter screen is prolonged.

The above embodiments are merely illustrative ones of the invention, and are not intended to limit the patent scope of the invention. All equivalent transformations made according to the contents in the specification and drawings of the invention, or direct or indirect applications to other relating technical fields should also fall within the patent protection scope of the invention.

What is claimed is:

1. A light shield structure, being disposed between an ultraviolet lamp and a filter screen of an air purifier, and comprising a first light shield element and a second light shield element;
the first light shield element is disposed on an inner wall of the second light shield element;
the second light shield element is provided with air inlets, the first light shield element is provided with air outlets, and the air inlets are staggered with the air outlets, so that the air outlets and the air inlets are completely blocked on at least one side in a radial direction; and an air inlet channel is formed between the first light shield element and the second light shield element.

2. The light shield structure according to claim 1, wherein first guide slopes and second guide slopes are disposed on an outer wall of the first light shield element, and a first angle which is less than 180° is formed between the first guide slopes and sides, away from the second light shield element, of the second guide slopes.

3. The light shield structure according to claim 1, wherein third guide slopes and fourth guide slopes are disposed on an outer wall of the second light shield element, and a second angle which is less than 180° is formed between the third guide slopes and sides, facing the first light shield element, of the fourth guide slopes.

4. The light shield structure according to claim 1, wherein the first light shield element is arranged on one side of the air inlets and is centered in a circumferential direction.

5. The light shield structure according to claim 1, further comprising multiple third light shield elements, wherein:
the multiple third light shield elements are regularly disposed on the inner wall of the second light shield element, and an air outlet channel is formed between every two adjacent said third light shield elements;
the air inlets and the air outlet channels opposite to the air inlets in the radial direction are communicated through the air inlet channel;
the first light shield element is arranged in the air inlet channel and is centered in the circumferential direction.

6. The light shield structure according to claim 5, wherein a fifth guide slope and a sixth guide slope are disposed on an outer wall of each said third light shield element, and a third angle which is less than 180° is formed between the fifth guide slope and a side, away from the second light shield element, of the sixth guide slope.

7. The light shield structure according to claim 4, wherein the first light shield element and the second light shield element are formed integrally.

8. The light shield structure according to claim 5, wherein the first light shield element and the second light shield element are formed integrally.

9. The light shield structure according to claim 6, wherein the first light shield element and the second light shield element are formed integrally.

10. The light shield structure according to claim 6, wherein the third light shield elements and the second light shield element are formed integrally.
